(19) United States
(12) Patent Application Publication
(10) Pub. No.: US 2026/0364311 A1
(43) Pub. Date: Nov. 19, 2026

(54) STEERABLE SHEATH HAVING DECREASED STIFFNESS REGION

(71) Applicant: Boston Scientific Scimed, Inc., Maple Grove, MN (US)

(72) Inventors: Matthew Paul Joseph DICICCO, Toronto (CA); Miles MONTGOMERY, East Gwillimbury (CA)

(21) Appl. No.: 19/675,952

(22) Filed: May 13, 2026

Related U.S. Application Data
(60) Provisional application No. 63/805,154, filed on May 13, 2025.

(51) Int. Cl. A61M 25/01; A61B 18/14; A61B 17/00

(52) U.S. Cl. CPC A61M 25/0138; A61B 18/1492; A61M 25/0136; A61B 2017/00247; A61B 2017/00323; A61M 2025/015

(57) ABSTRACT

A steerable sheath includes an elongated member having a distal end, a proximal end and a shaft defining a lumen extending between the proximal end and the distal end, wherein the lumen is configured for receiving a catheter therein. The steerable sheath also includes a steering wire comprising a distal end coupled to a first lateral side of the elongated member, wherein tensioning the first steering wire bends the distal end of the elongated member in a first direction to create a first bending configuration. The shaft on the first lateral side of the elongated member proximal from the steering wire coupling pull ring is stiffer that the shaft diametrically opposite on a second lateral side of the elongated member.

### CROSS REFERENCE TO RELATED APPLICATIONS

The present application claims priority to U.S. Provisional Patent Application No. 63/805,154, filed May 13, 2025, the entire disclosure of which is incorporated herein in its entirety.

### TECHNICAL FIELD

The present disclosure relates to medical systems and methods for steering medical devices in a patient. More specifically, the present disclosure relates to steerable sheaths.

### BACKGROUND

Various medical fields use different types of catheters to achieve access to a physiological site in medical procedures. For instance, electrophysiological procedures involve guiding catheter elements into the heart and tracking the location of the catheters with respect to the heart. Catheter ablation is a minimally invasive electrophysiological procedure to treat a variety of heart conditions such as supraventricular and ventricular arrhythmia. Example catheter elements used in catheter ablation can include mapping catheters, ablation catheters, guiding sheaths or introducer sheaths, dilators, and other medical tools, which can be referred to as catheters in this disclosure.

To position a catheter element within the body at a target site, some type of navigation mechanism is used, such as a steering feature incorporated into the catheter. Clinicians manually manipulate or operate the catheter using the steering feature. To facilitate the advancement of catheters through a patient's vasculature, the concurrent application of torque at a proximal portion of the catheter and the ability to deflect the distal portion of the catheter in a selected direction permits the clinician to adjust the direction of advancement of the distal portion of the catheter and to selectively position the distal portion of the catheter during an electrophysiological procedure. In an example, the distal portion includes a pull ring assembly having a pull ring that is deflected with a steering wire or other tension member attached or anchored at the distal end of the catheter and extending proximally to an actuator in a control handle that controls the application of tension on the steering wire. Often, a catheter element is configured to deflect in more than one direction and includes one or more steering wires installed on the pull ring on opposite side from each other. For example, the catheter can include a pair of steering wires or two pairs of steering wires for four-way steering.

### SUMMARY

In Example 1, a steerable sheath, comprising: an elongated member having a distal end, a proximal end and a shaft defining a lumen extending between the proximal end and the distal end, wherein the lumen is configured for receiving a catheter therein; and a steering wire comprising a distal end coupled to a first lateral side of the elongated member, wherein tensioning the first steering wire bends the distal end of the elongated member in a first direction to create a first bending configuration; wherein the shaft on the first lateral side of the elongated member proximal from the steering wire coupling pull ring is stiffer that the shaft diametrically opposite on a second lateral side of the elongated member.

In Example 2 the sheath of Example 1, wherein the difference in stiffness between the first lateral side and the second lateral side is achieved through different durometer polymers.

In Example 3 the sheath of Example 1, wherein the difference in stiffness between the first lateral side and the second lateral side is achieved through varying polymer thickness

In Example 4 the sheath of Example 1, wherein the difference in stiffness between the first lateral side and the second lateral side is achieved through asymmetric braiding or coiling.

Example 5 the sheath of Example 1, wherein the difference in stiffness between the first lateral side and the second lateral side is achieved through braid welding or coil welding.

In Example 6 the sheath of Example 1, wherein the difference in stiffness between the first lateral side and the second lateral side is achieved through cutouts in the shaft.

In Example 7 the sheath of Example 1, further comprising a second steering wire coupled to the second lateral side of the elongated member, wherein the elongated member is configured for bi-directional steering with asymmetric curves.

In Example 8 the sheath of Example 7, wherein the first bending configuration comprises a substantially open arch curved profile having a first predetermined circumference, and the second bending configuration comprises a sharply curved profile having a second predetermined circumference smaller than the first predetermined circumference.

In Example 9 the sheath of Example 1, wherein the elongated member includes a pre-curved portion configured to increase reach on one side while decreasing reach on the opposite side.

In Example 10 the sheath of Example 1, further comprising a handle coupled to the proximal end of the elongated member and a steering mechanism mounted to the handle, wherein a proximal end of the first steering wire is coupled to the steering mechanism.

In Example 11 the sheath of Example 1, wherein the elongated member is configured to produce a first bending configuration when the steerable catheter is unaffected which is different from a shape of a second bending configuration when force is exerted on the elongated member proximal to the steering wire coupling.

In Example 12 the sheath of Example 1, wherein the first bending configuration is an S-curve.

In Example 13 the sheath of Example 12, wherein the S-curve is configured to reduce a distance between the distal end and a midline of the sheath to increase working space for maneuvering a catheter received within the lumen.

In Example 14 the sheath of Example 1, wherein the elongated member is configured to access pulmonary veins through a transseptal puncture in a patient's heart.

In Example 15 the sheath of Example 1, wherein the difference of stiffness between the first lateral side and the second lateral side is about 1.5 to about 5.0 times.

In Example 16, a steerable sheath, comprising: an elongated member having a distal end, a proximal end and a shaft defining a lumen extending between the proximal end and the distal end, wherein the lumen is configured for receiving a catheter therein; and a steering wire comprising a distal end coupled to a first lateral side of the elongated member, wherein tensioning the first steering wire bends the distal end of the elongated member in a first direction to create a first bending configuration; wherein the shaft on the first lateral side of the elongated member proximal from the steering wire coupling pull ring is stiffer that the shaft diametrically opposite on a second lateral side of the elongated member.

In Example 17 the sheath of Example 16, wherein the difference in stiffness between the first lateral side and the second lateral side is achieved through different durometer polymers.

In Example 18 the sheath of Example 16, wherein the difference in stiffness between the first lateral side and the second lateral side is achieved through varying polymer thickness.

In Example 19 the sheath of Example 16, wherein the difference in stiffness between the first lateral side and the second lateral side is achieved through asymmetric braiding or coiling.

In Example 20 the sheath of Example 16, wherein the difference in stiffness between the first lateral side and the second lateral side is achieved through cutouts in the shaft.

In Example 21, the sheath of Example 16, further comprising a second steering wire coupled to the second lateral side of the elongated member, wherein: the elongated member is configured for bi-directional steering with asymmetric curves the first bending configuration comprises a substantially open arch curved profile having a first predetermined circumference; and the second bending configuration comprises a sharply curved profile having a second predetermined circumference smaller than the first predetermined circumference.

In Example 22 the sheath of Example 16, wherein the elongated member includes a pre-curved portion configured to increase reach on one side while decreasing reach on the opposite side.

In Example 23 the sheath of Example 16, further comprising a handle coupled to the proximal end of the elongated member and a steering mechanism mounted to the handle, wherein a proximal end of the first steering wire is coupled to the steering mechanism.

In Example 24, a steerable sheath, comprising: an elongated member having a distal end, a proximal end and a shaft defining a lumen extending between the proximal end and the distal end, wherein the lumen is configured for receiving a catheter therein; a steering wire comprising a distal end coupled to a first lateral side of the elongated member, wherein tensioning the first steering wire bends the distal end of the elongated member in a first direction; wherein the shaft comprises a first lateral side having a first stiffness and a second lateral side having a second stiffness, wherein the first stiffness is greater than the second stiffness; and wherein the elongated member is configured to form an S-curve when a force is exerted on the shaft proximal to where the steering wire is coupled.

In Example 25 the steerable sheath of Example 24, wherein the difference in stiffness between the first lateral side and the second lateral side is achieved through different durometer polymers.

In Example 26 the steerable sheath of Example 24, wherein the difference in stiffness between the first lateral side and the second lateral side is achieved through varying polymer thickness.

In Example 27 the steerable sheath of Example 24, wherein the difference in stiffness between the first lateral side and the second lateral side is achieved through asymmetric braiding or coiling.

In Example 28, the steerable sheath of Example 24, further comprising a second steering wire coupled to the second lateral side of the elongated member, wherein: the elongated member is configured for bi-directional steering with asymmetric curves; a first bending configuration comprises a substantially open arch curved profile having a first predetermined circumference; and a second bending configuration comprises a sharply curved profile having a second predetermined circumference smaller than the first predetermined circumference.

In Example 29 the steerable sheath of Example 24, wherein the elongated member includes a pre-curved portion configured to increase reach on one side while reducing reach on the opposite side.

In Example 30 the steerable sheath of Example 24, further comprising a handle coupled to the proximal end of the elongated member and a steering mechanism mounted to the handle, wherein a proximal end of the first steering wire is coupled to the steering mechanism.

In Example 31 the steerable sheath of Example 24, wherein the S-curve is configured to reduce a distance between the distal end and a midline of the sheath to increase working space for maneuvering a catheter received within the lumen.

In Example 32 the steerable sheath of Example 24, wherein the elongated member is configured to access pulmonary veins through a transseptal puncture in a patient's heart.

In Example 33 a method of treating a patient, comprising: a steerable sheath into a patient's vasculature, the steerable sheath comprising an elongated member having a distal end and a shaft defining a lumen, wherein the shaft comprises a first lateral side having a first stiffness and a second lateral side having a second stiffness less than the first stiffness; advancing a catheter through the lumen of the elongated member; tensioning a steering wire coupled to the first lateral side to bend the distal end; and positioning the distal end such that anatomical forces cause the elongated member to form an S-curve that pulls the distal end toward a midline of the elongated member.

In Example 34 the method of Example 33, further comprising performing a transseptal puncture and advancing the distal end through the puncture to access pulmonary veins.

In Example 35 the method of Example 34, further comprising performing an ablation procedure to treat cardiac arrhythmia.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is an exemplary elongated catheter assembly showing a catheter and steerable catheter sheath, with the catheter configured to be inserted through a lumen in the sheath from the proximal end toward the distal end.

FIG. 2B is a cut-away view of the elongated member showing the steering wire coupled to a lateral side via a ring.

FIG. 2C is a view showing the distal portion of the elongated member in two different bending configurations - a first configuration with a substantially open arch curved profile and a second configuration with a sharply curved profile.

FIG. 3A is a diagram showing the steerable catheter sheath accessing left and right pulmonary veins through the septum.

FIG. 3B is a diagram showing the steerable catheter sheath accessing the left atrial appendage.

FIG. 4A is a view of the steerable catheter sheath showing the first lateral side and second lateral side, with the first lateral side having greater stiffness than the second lateral side.

FIG. 4B is a view showing how force exerted on the sheath results in the elongated member forming an S-curve for stress relief.

FIGS. 5A and 5B are comparative views showing the increased working space for maneuvers when a stress relief feature is provided, with FIG. 5A showing a prior art sheath without stress relief, and FIG. 5B showing a sheath with stress relief that reduces the distance between the distal tip and the midline.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

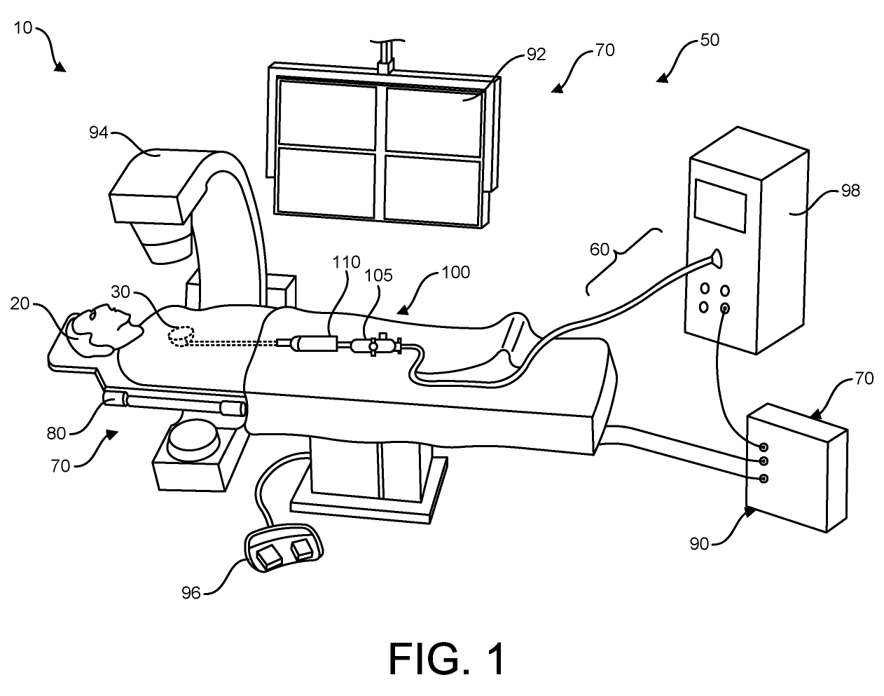

FIG. 1 is a diagram illustrating an exemplary clinical setting 10 for treating a patient 20, and for treating a heart 30 of the patient 20, using an electrophysiology system 50 includes a catheter system. The example catheter system 60 includes an elongated catheter assembly 100, which in the example, includes a catheter 105 and a steerable catheter sheath 110 and a treatment console 98 such as an ablation or electroporation console. Additionally, the catheter system 60 includes various connecting elements, such as cables, that operably connect the components of the catheter system 60 to one another and to the treatment console 98. Also, the clinical setting 10 can include additional equipment such as an electro anatomical mapping (EAM) system 70 including a localization field generator 80, a mapping and navigation controller 90, and a display 92. The clinical setting can further include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

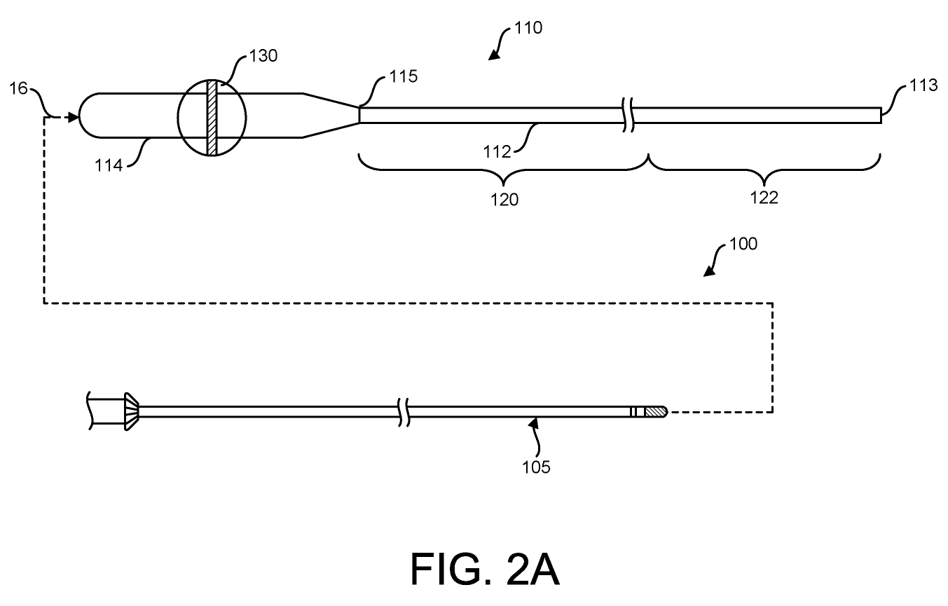

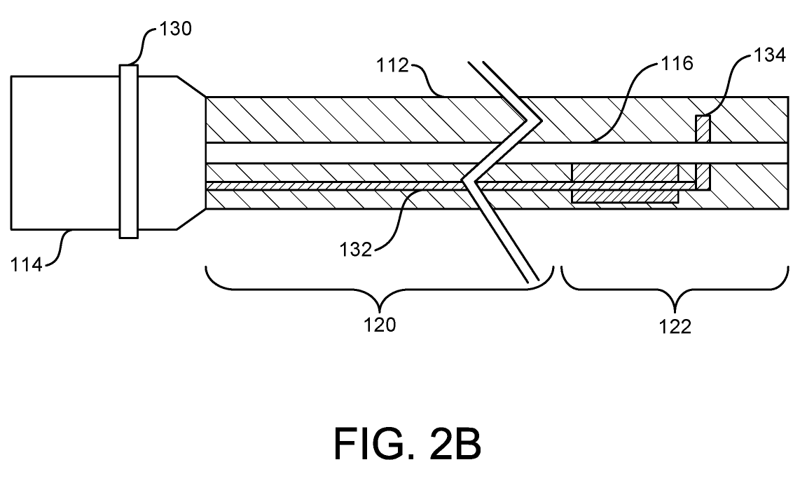

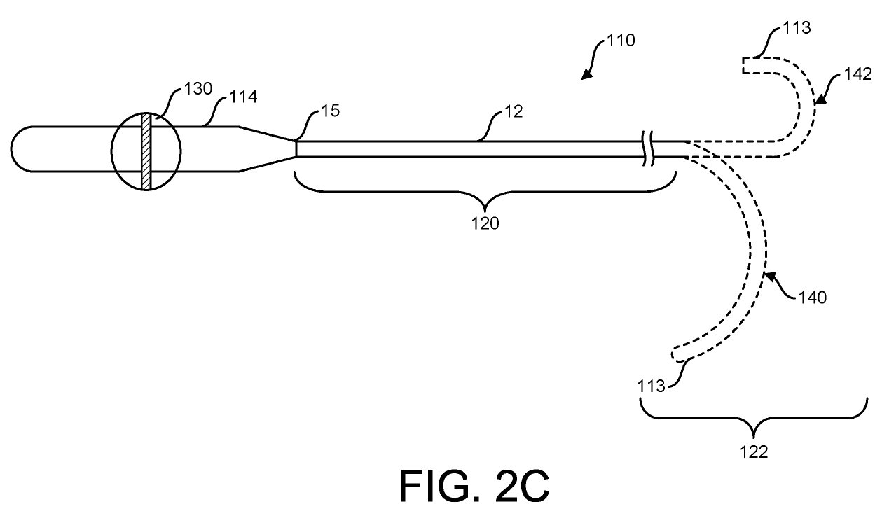

Referring to FIG. 2A, an exemplary elongated catheter assembly 100 includes a catheter 105 and a steerable catheter sheath 110 sized for receiving the catheter 105 therein. In the illustrated embodiment, the catheter 105 is an ablation catheter or a mapping/ablation catheter. The catheter assembly 100 may further include a guide wire and/or other therapeutic tools (not shown) to be delivered into the left atrium in a desired position.

Although the steerable catheter sheath 110 is described hereinafter for use in the heart for facilitating introduction of the catheter 105 into desirable positions for mapping and ablating arrhythmia substrates, the sheath 110 may be used within any body lumens, chambers or cavities of a patient for therapeutic and diagnostic purposes in those instances where access to interior bodily regions is obtained through, for example, the vascular system or alimentary canal and without complex invasive surgical procedures.

The steerable catheter sheath 110 generally comprises an elongated member 112, a handle 114 coupled to a proximal end 115 of the elongated member 112, and a lumen 116 extending through the elongated member 112 for allowing the catheter 105, guide wire (not shown) and/or other therapeutic tools (not shown) to be inserted from a proximal end of the handle 114 towards a distal end 113 of the elongated member 112. (as indicated by arrow 16 in FIG. 2A). The elongated member 112 includes a proximal portion 120 and a steerable distal portion 122.

The distal end 113 of the elongated member 112 is configured to be introduced through the vasculature of the patient, and into an anatomical cavity, such as the left atrium of the heart. The distal portion 122 is configured to selectively bend. The proximal portion 120 of the elongated member 112 is formed of a composite of PEBAX and stainless steel or other suitable materials, in the form of a wire, braid, coil, or combination thereof, so that the proximal portion 120 of the elongated member 112 has desired stiffness and torsional properties. In some embodiments, the distal portion 122 of the elongated member 112 is formed of a more flexible material so that the distal portion 122 has desired bending properties. The sheath 110 further comprises a steering mechanism 130 incorporated into the handle 114.

As shown in cut-away view of FIG. 2B, a steering wire 132 extends through the elongated member 112. The distal end of the steering wire 132 coupled to a lateral side of the elongated member 112. The steering mechanism 130 is operable to selectively tension the steering wire 132, thereby transforming the distal portion 122 of the elongated member 112 from a straight geometry to a bending configuration. In the embodiment of FIG. 2B, the steering wire 132 is coupled to the lateral side of the elongated member 112 via a ring 134.

The specific embodiment of shown in FIG. 2C comprises a pair of steering wires 132 which extend along diametrically opposed locations through the elongated member 112. The selective tensioning of the steering wires 132 by the steering mechanism 130 transforms the distal portion of the elongated member 112 from its straight geometry shown in FIG. 2A, into one of its bending configurations, for example, bending configurations 140, 142, shown in FIG. 2C. The proximal ends of the steering wires 132 are coupled to the steering mechanism 130 in the handle 114, while the distal ends of the steering wires 132 are affixed within the distal portion 122 of the elongated member 112. In one embodiment, the distal ends of the steering wires 132 are welded or otherwise affixed to a pull ring 134 that is disposed within the distal portion 122. In particular, one of the steering wires 132 is coupled to a first lateral side of the elongated member 112 and the other steering wire 132 is coupled to a second lateral side of the elongated member 112. In one embodiment, the first and second lateral sides of the elongated member 112 are diametrically opposed. In one embodiment, the steering wires 132 are affixed to opposing sides of the pull ring 134. In an alternative embodiment, the steering wires 132 may be attached at different longitudinal positions along the elongated member 112, in which case the elongated member 112 may include two pull rings 134 wherein the steering wires 132 are each attached to different pull rings 134.

In the embodiment shown in FIG. 2C, the distal portion 122 is configured to selectively bend in two directions, each bending direction having its own specific configuration. The curvature assumed by bending the distal portion 122 to the right is different from the curvature assumed by bending the distal portion 122 to the left. For instance, when deflected to the right, the distal portion 122 assumes a first configuration 140, e.g., a substantially open arch curved profile with a first predetermined circumference. In contrast, when deflected to the left, the distal portion 122 assumes a second configuration 142, e.g., a sharply curved profile with a second predetermined circumference. These different left and right curvatures are referred herein as asymmetric curves.

In an exemplary embodiment, the second configuration 142 has a substantially smaller radius of curvature than that of the first configuration 140. The asymmetric bending of the distal portion 122 results from different bending properties on the respective lateral sides of the distal portion 122 of the elongated member 112. The specific circumferences, radii of curvature, and shapes that the distal portion 122 will assume is related to parameters such as the size shape, material of construction, and location of various elements of the elongated member 112. It should be well understood that any desired circumference, radius of curvature, and shape of the two bent configurations 140, 142 may be achieved by adjusting such parameters.

Cardiac ablation procedures are commonly performed to treat various cardiac arrhythmias by creating therapeutic lesions in the heart tissue. During these procedures, catheters are introduced into the heart chambers through the vasculature to map electrical signals and deliver ablation energy to specific target sites. Access to the left atrium is typically achieved through a transeptal approach where instruments are passed from the right atrium through the atrial septum. During these procedures, the anatomy targets result in a reduced working space to maneuver the steerable catheter sheath 110 and the catheter 105.

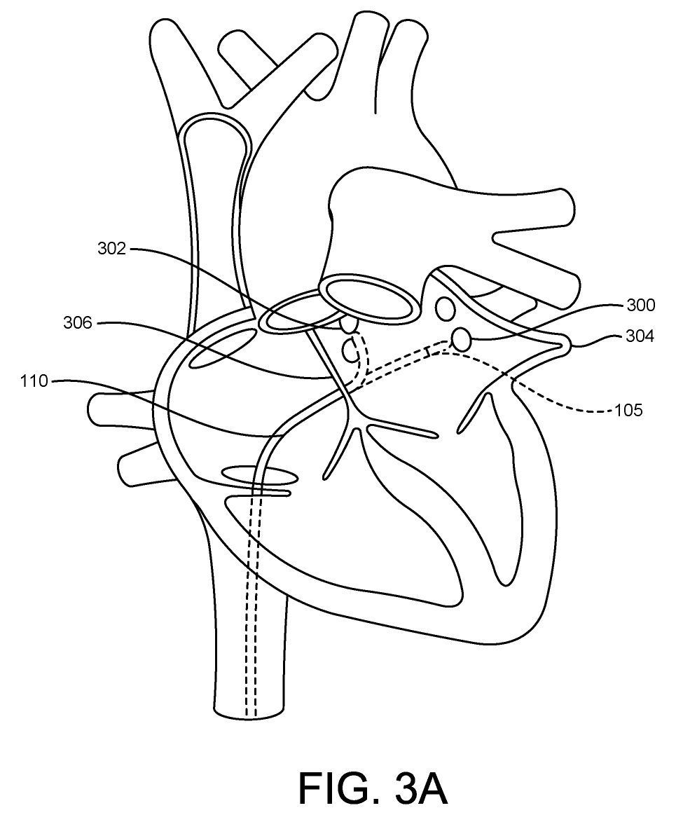

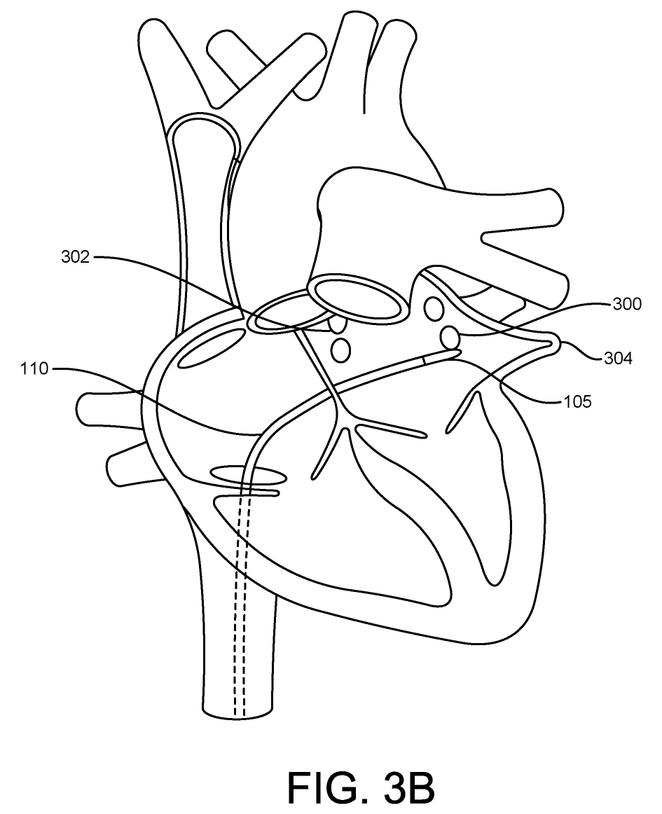

As shown in FIG. 3A and 3B, when performing procedures in the left atrium, physicians must navigate catheters to reach different anatomical targets including the pulmonary veins, where many arrhythmia substrates are located. In a specific embodiment, the reach needed to support an ablation at the left pulmonary veins restricts the ability to maneuver the catheter to the right pulmonary veins. The specific anatomical arrangement of left and right pulmonary veins present distinct access challenges, as the right and left pulmonary veins are positioned at different orientations relative to the transeptal access point. FIG. 3A shows an embodiment of the steerable catheter sheath 110 accessing the left pulmonary veins 300 and the right pulmonary veins 302 through the septum 306. FIG. 3B shows an alternate procedure where the steerable sheath 110 additionally accesses the left atrial appendage 304. Some procedures combine the ablation of the pulmonary veins with left atrial appendage occlusion. In such procedures, the location of the transeptal puncture will impact the sheath 110 positioning for one or both procedures.

Positioning of the sheath 110 and therapeutic tools (not shown) is critical to the success of the procedure. The steerable sheath 110 must be maneuvered differently when placing the catheter in contact with the left pulmonary veins as opposed to the right pulmonary veins. Additionally, some therapy catheters 105, such as basket catheters for ablation, require additional space for maneuverability. In these cases, it is beneficial for the distal tip of the sheath 110 to be spaced away from the target location for the deployment of the therapeutic catheter. Pulling the sheath tip 113 away from the anatomical targets can improve the maneuvering of the therapy catheter and provide the required space for navigation or deployment of the therapy catheter 105.

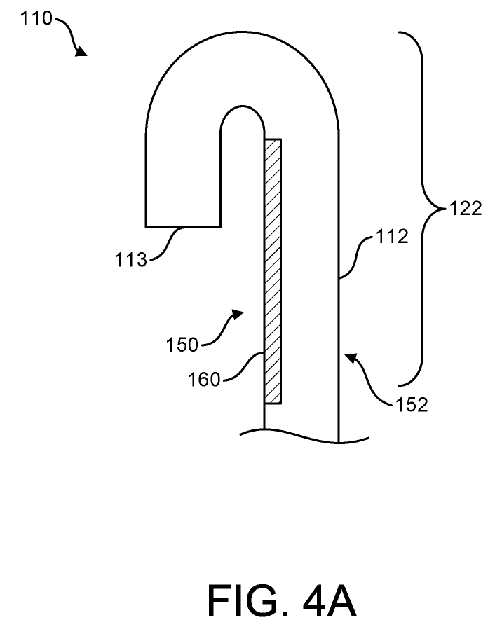

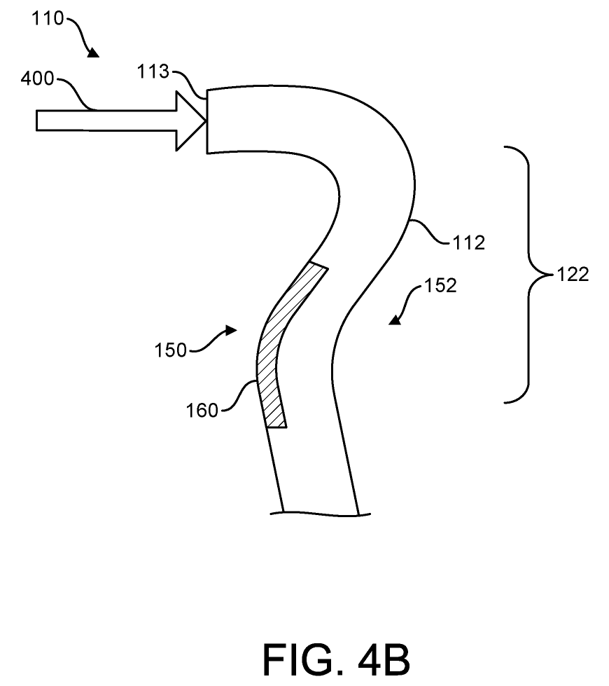

FIG. 4A illustrates an embodiment of a steerable catheter sheath 110 including distal portion 122 configured to pull away from anatomical targets to improve maneuvering and to improve the accessibility for treatment. In a specific embodiment, the stiffness delta on opposing sides of the curve results in stress relief of the curve to improve reach. FIG. 4A shows a first lateral side 150 and a second lateral side 152. The first lateral side 150 comprises a portion of the shaft of the elongated member 112 proximal from the steering wire pull ring 134 which is stiffer than the shaft of the elongated member 112 on the opposing second lateral side 152. The difference in relative stiffness between the first lateral side 150 and the second lateral side 152 may be a result of increased stiffness of the first lateral side 150, or a decreased stiffness of the second lateral side 152. In various embodiments, the first lateral side 150 has a stiffness of from about 1.5 to about 5.0 times greater that a stiffness of the second lateral side.

In one embodiment, such relative stiffness is achieved by modifying the area 160. I n various embodiments, the area 160 has a length of between about 3 and about 10cm.In certain embodiments, to achieve this relative stiffness, the polymer is thicker on the first lateral side 150 in the area 160. This may be achieved through an additive process. Alternatively, the second lateral side 152 may have some polymer removal to reduce the polymer thickness. In an alternate embodiment, the material chosen on the first lateral side 150 in the area 160 may be a stiffer material (e.g., having a higher flexural modulus) than the second lateral side 152. This may be achieved by removing the material in area 160 of the elongated member 112 and adding in material with a higher durometer which is then reflowed to provide as smooth transition on the outer surface. Alternatively, this process may be done on the second lateral side 152 wherein the new material inserted has a lower durometer and then reflowed. In a further alternative, both the first lateral side 150 and the second lateral side 152 have material removed wherein the added material on the first lateral side 150 has a higher durometer than the added material to the second lateral side 152. In another alternate embodiment, the area 160 is stripe extruded wherein a higher durometer material is co-extruded on the first lateral side 150 while a lower durometer material is co-extruded on the second lateral side 152.

In another alternate embodiment, a braid or coil (not shown) is positioned within the shaft of the elongated member 112 having a braid or coil stiffness greater on one side via braid welding, coil, welding, or coil pitch. In a further alternate embodiment, the shaft of the elongated member 112 comprises a hypotube. The hypotube may be laser cut (not shown) to provide articulation joints to reduce the stiffness of the second lateral side 152.

The differing stiffness between the first lateral side 150 and the second lateral side 152 provides a stress relief feature of the elongated member 112. The stress relief in the distal portion 122 of the elongated member 112 can improve the reach of the steerable sheath 110 allowing the distal tip 113 to pull away from the target tissue.

FIG. 4B illustrates the stress relief of the elongated member 112 when a force, depicted by arrow 400, is exerted on the sheath 110. As the second lateral side 152 has a lower stiffness than the first lateral side 150, force 400 exerted during a procedure (for example the force exerted via the septum after a transeptal puncture) results in the elongated member 112 forming an S-curve for stress relief. The shape of the S-curve at a given quantity of the force 400 can be adjusted by changing the properties of the elongated member in the area 160. For example, if the area 160 is stripe extruded, changing the widths of the stripes can alter the deflection and stress relief provided.

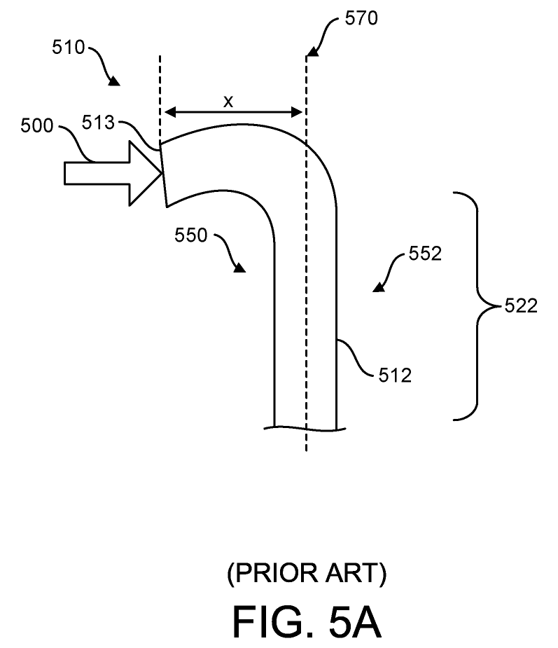

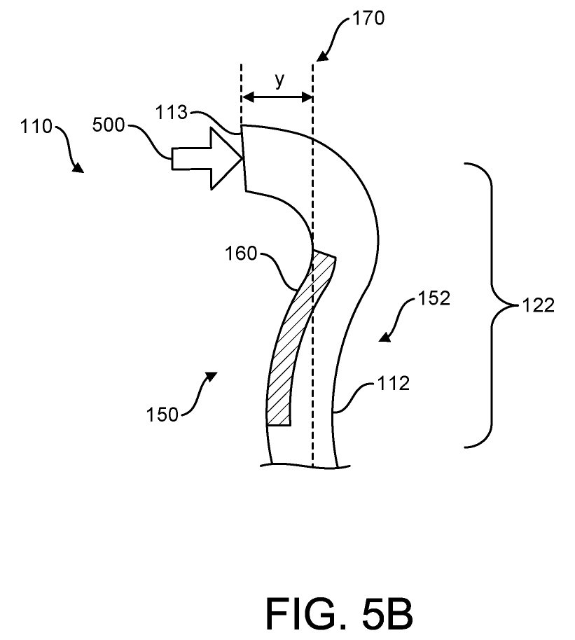

FIGS. 5A and 5B illustrate the increased working space for maneuvers when a stress relief feature is provided. FIG. 5A shows a prior art steerable sheath 510 without a stress relief feature. The first lateral side 550 and the second lateral side 552 of the distal portion 522 of the sheath 510 have the same stiffnesses. As a force (arrow 500) is exerted on the elongated member 512, the shaft of the elongated member 112 remains substantially aligned with the midline 570 of the sheath 510. The reach of the sheath is shown as the distance x which is measured from the midline 570 of the sheath 510 to the distal tip 513 of the sheath 510. FIG. 5B shows a steerable sheath 110 having a stress relief feature in the area 160. In this embodiment, the first lateral side 150 has a higher stiffness than the second lateral side 152. As a force (arrow 500) is exerted on the elongated member 112, the stress relief feature results in the shaft of the elongated member 112 to bend towards the side of reduced stiffness (i.e. the second lateral side 152). This stress relief results in an S-curve wherein the shaft of the elongated member 112 is offset from the midline 170 of the sheath 110. In other words, the distal tip 113 is pulled towards the midline 170 of the sheath 110. The reach of the sheath 110 is shown as the distance y which is measured from the midline 170 of the sheath 110 to the distal tip 113 of the sheath 110. Due to the s-curve induced by the stress relief feature, the reach y of sheath 110 is less than the reach x of the sheath 510 without the stress relief feature. Therefore, the sheath 110 having the stress relief feature increases the working space for maneuvers.

The increased working space created by the sheath 110 may allow for easier access to particular target tissues or may provide room for therapy catheters which require space to deploy or change shape. Furthermore, the increased space may allow for larger or more complex form factors of the catheter 105. The improved access, maneuverability, and therapy catheter form factors can streamline procedures and reducing the time required for a procedure. The improved maneuverability from the sheath 110 with a stress relief feature can also reduce the sensitivity of the transseptal puncture location. With more flexibility with the transseptal position, the physician can reduce the time required for performing the transseptal punction prior to mapping or treatment. The reduced time for a transseptal puncture reduces the overall time of the procedure and simplifies the process for the physician. The sheath 110 with stress relief features that reduce reach allows the physician more space to maneuver, allows for easier access to a variety of anatomical landmarks (left and right pulmonary veins, left atrial appendage, etc.), and allows more variability in the transseptal puncture location improving the ease of use of the mapping or therapy catheter and improving the workflow of the procedure.

In some embodiments, the sheath 110 is pre-curved to provide further stress relief. In such embodiments, the sheath 110 has curved set during the manufacturing process. When in use, as the force is exerted to the area 160, the stress relief results in a more pronounced S-curve. This amplified S-curve decreases the distance y between the distal end 113 and the midline 170 of the sheath 110 further increasing the working space for the catheter 105. The pre-curved portion increases the reach on one side while reducing the reach on the opposite side.

In a further alternate embodiment, the sheath 110 has bi-directional steering. As shown in FIG. 2C, the sheath 110 may have asymmetric curves. The combination of the sheath 110 having a stress relief feature and asymmetric steerable curves allows increased maneuverability. In concomitant interventions where various anatomical landmarks may be targeted, the asymmetric curves allow further movement to the target site. In a specific procedure, the sheath 110 would be curved in one direction to occlude the left atrial appendage (FIG. 3B) and be curved in the opposite direction to access the left and right pulmonary veins (FIG. 3A). The stress relief feature of the sheath 110 allows for more flexibility in the placement of the transseptal puncture to allow access to the opposing anatomical locations thereby simplifying the procedure. The asymmetric curves have a first bending configuration that has a substantially open arch curved profile with a first predetermined circumference, and a second bending configuration having a sharply curved profile with a second predetermined circumference smaller than the first predetermined circumference.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A steerable sheath, comprising:
an elongated member having a distal end, a proximal end and a shaft defining a lumen extending between the proximal end and the distal end, wherein the lumen is configured for receiving a catheter therein; and
a steering wire comprising a distal end coupled to a first lateral side of the elongated member, wherein tensioning the first steering wire bends the distal end of the elongated member in a first direction to create a first bending configuration;
wherein the shaft on the first lateral side of the elongated member proximal from the steering wire coupling pull ring is stiffer that the shaft diametrically opposite on a second lateral side of the elongated member.

2. The sheath of claim 1, wherein the difference in stiffness between the first lateral side and the second lateral side is achieved through different durometer polymers.

3. The sheath of claim 1, wherein the difference in stiffness between the first lateral side and the second lateral side is achieved through varying polymer thickness.

4. The sheath of claim 1, wherein the difference in stiffness between the first lateral side and the second lateral side is achieved through asymmetric braiding or coiling.

5. The sheath of claim 1, wherein the difference in stiffness between the first lateral side and the second lateral side is achieved through cutouts in the shaft.

6. The sheath of claim 1, further comprising a second steering wire coupled to the second lateral side of the elongated member, wherein:
the elongated member is configured for bi-directional steering with asymmetric curves
the first bending configuration comprises a substantially open arch curved profile having a first predetermined circumference; and
the second bending configuration comprises a sharply curved profile having a second predetermined circumference smaller than the first predetermined circumference.

7. The sheath of claim 1, wherein the elongated member includes a pre-curved portion configured to increase reach on one side while decreasing reach on the opposite side.

8. The sheath of claim 1, further comprising a handle coupled to the proximal end of the elongated member and a steering mechanism mounted to the handle, wherein a proximal end of the first steering wire is coupled to the steering mechanism.

9. A steerable sheath, comprising:
an elongated member having a distal end, a proximal end and a shaft defining a lumen extending between the proximal end and the distal end, wherein the lumen is configured for receiving a catheter therein;
a steering wire comprising a distal end coupled to a first lateral side of the elongated member, wherein tensioning the first steering wire bends the distal end of the elongated member in a first direction;
wherein the shaft comprises a first lateral side having a first stiffness and a second lateral side having a second stiffness, wherein the first stiffness is greater than the second stiffness; and
wherein the elongated member is configured to form an S-curve when a force is exerted on the shaft proximal to where the steering wire is coupled.

10. The steerable sheath of claim 9, wherein the difference in stiffness between the first lateral side and the second lateral side is achieved through different durometer polymers.

11. The steerable sheath of claim 9, wherein the difference in stiffness between the first lateral side and the second lateral side is achieved through varying polymer thickness.

12. The steerable sheath of claim 9, wherein the difference in stiffness between the first lateral side and the second lateral side is achieved through asymmetric braiding or coiling.

13. The steerable sheath of claim 9, further comprising a second steering wire coupled to the second lateral side of the elongated member, wherein:
the elongated member is configured for bi-directional steering with asymmetric curves;
a first bending configuration comprises a substantially open arch curved profile having a first predetermined circumference; and
a second bending configuration comprises a sharply curved profile having a second predetermined circumference smaller than the first predetermined circumference.

14. The steerable sheath of claim 9, wherein the elongated member includes a pre-curved portion configured to increase reach on one side while reducing reach on the opposite side.

15. The steerable sheath of claim 9, further comprising a handle coupled to the proximal end of the elongated member and a steering mechanism mounted to the handle, wherein a proximal end of the first steering wire is coupled to the steering mechanism.

16. The steerable sheath of claim 9, wherein the S-curve is configured to reduce a distance between the distal end and a midline of the sheath to increase working space for maneuvering a catheter received within the lumen.

17. The steerable sheath of claim 9, wherein the elongated member is configured to access pulmonary veins through a transseptal puncture in a patient's heart.

18. A method of treating a patient, comprising:
introducing a steerable sheath into a patient's vasculature, the steerable sheath comprising an elongated member having a distal end and a shaft defining a lumen, wherein the shaft comprises a first lateral side having a first stiffness and a second lateral side having a second stiffness less than the first stiffness;
advancing a catheter through the lumen of the elongated member;
tensioning a steering wire coupled to the first lateral side to bend the distal end; and
positioning the distal end such that anatomical forces cause the elongated member to form an S-curve that pulls the distal end toward a midline of the elongated member.

19. The method of claim 18, further comprising performing a transseptal puncture and advancing the distal end through the puncture to access pulmonary veins.

20. The method of claim 18, further comprising performing an ablation procedure to treat cardiac arrhythmia.
